(19) United States
(12) Patent Application Publication
(10) Pub. No.: US 2005/0074488 A1
(43) Pub. Date: Apr. 7, 2005

(54) Use of vitamin Ds to down regulate the renin-angiotensin-aldosterone system

(76) Inventors: Joel Melnick, Wilmette, IL (US); Jin Tian, Waukegan, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/900,418

(22) Filed: Jul. 27, 2004

Related U.S. Application Data
(60) Provisional application No. 60/490,478, filed on Jul. 28, 2003.

(51) Int. Cl. A61K 31/59; A61K 9/70

(52) U.S. Cl. 424/449; 514/167

(57) ABSTRACT

The present invention relates to the use of Vitamin D, preferably paricalcitol, to treat, prevent and delay disease progression of diseases associated with over activation of the renin-angiotensin aldosterone system.

The present application claims priority to U.S. Provisional Application No. 60/490,478, filed on Jul. 30, 2003, hereby incorporated in its entirety by reference.

### BACKGROUND INFORMATION

1. Field of the Invention

The present invention relates to the use of a Vitamin D, preferably paricalcitol, to treat, prevent and delay progression of diseases associated with over-activation of the renin-angiotensin-aldosterone system.

2. Background of the Invention

Angiotensin-converting-enzyme inhibitors (ACEI) are known to inhibit the renin-angiotensin-aldosterone system (RAAS) pathway. Angiotensin II (AII) receptor 1 blockers (ARBs) and aldosterone inhibitors can synergistically inhibit the effects of the RAAS pathway. An important limitation to long term use of ACEIs, ARBs and aldosterone inhibitors is that these increase upstream proteins, e.g., renin, in the RAAS pathway, leading to ACE escape and increased angiotensin II and aldosterone production.

There are no commercially available renin inhibitors to treat over-activation of RAAS, especially any that are suitable for long term use.

All patents and publications referred to herein are hereby incorporated in their entirety by reference.

### SUMMARY OF THE INVENTION

The present invention is directed to methods for preventing, treating and delaying progression of diseases caused by the pathogenic effects of increased angiotensin II or renin in the body.

In one aspect of the invention, a Vitamin D or Vitamin D analog is administered to a patient in need of treatment to synergistically down regulate RAAS by inhibiting the upstream protein, renin, which would be expected to prevent ACE/aldosterone escape. Thus, according to one embodiment, the present invention relates to Vitamin D or Vitamin D analog-containing compositions for preventing, treating and delaying progression of such diseases. Compositions according to the invention would suitably contain a Vitamin D or Vitamin D analog and at least one of the following agents: angiotensin-converting-enzyme inhibitor, angiotensin II receptor 1 blocking agent (ARB) and aldosterone blocking agent.

According to some aspects of the present invention, the Vitamin D or Vitamin D analog is paricalcitol or a paricalcitol analog. Paricalcitol is commercially available from Abbott Laboratories (North Chicago, Ill., under the tradename ZEMPLAR). Other Vitamin D or Vitamin D analogs include but are not limited to calcitriol (e.g., CALCIJEX brand, available from Abbott Laboratories of North Chicago, Ill.) and doxercalciferol. Especially preferred compositions of the present invention also include one or more of the following agents: an angiotensin-converting-enzyme inhibitor (ACEI) or an angiotensin II receptor 1 (AT1) blocker or an aldosterone blocker (ARB). According to other aspects of the invention, pharmaceutical compositions can be administered through a sustained (or continuous) delivery system. The present invention also contemplates other modes of administration, including but not limited to oral, injectable and transdermal.

### BRIEF DESCRIPTION OF THE DRAWINGS

FIG. 1 illustrates a Northern blot which evidences that paricalcitol treatment of As4.1-hVDR cells dose-dependently inhibits renin mRNA expression.

FIG. 2 illustrates the results of a renin promoter-luciferase assay used to examine the activity of paricalcitol to suppress renin gene transcription.

FIG. 3 illustrates VDR expression in normal human tissues.

FIG. 4 illustrates the effect of paricalcitol and calcitriol on PAI-1 in primary culture of human coronary artery smooth muscle cells.

### DETAILED DESCRIPTION OF THE INVENTION

The present invention is generally directed to compositions containing a Vitamin D or Vitamin D analog to treat, prevent or delay disease progression due to over-activation of the renin-angiotensin-aldosterone system. The present invention also relates to methods of treating these diseases by administering to a patient a pharmaceutical composition containing a therapeutically effective amount of a Vitamin D or Vitamin D analog.

Treatment of patients at risk for or already having these diseases according to the invention would be accomplished by administration of a therapeutically effective amount of a Vitamin D or Vitamin D analog-containing composition is expected to be advantageous for effective reduction of renin expression. In contrast, conventional treatments based on administration of an ACEI (i.e., without a Vitamin D or Vitamin D analog) for example only reduce angiotensin II, but do not reduce renin levels or act on Vitamin D receptors in the body itself. Administration of ACEI may not an attractive long term treatment due to adverse consequences.

According to some aspects of the present invention, the inventive compositions contain a Vitamin D or Vitamin D analog and at least one of the following agents: an ACE inhibitor, an angiotensin II receptor blocker (ARB) and aldosterone blocker in therapeutically effective amounts to inhibit renin production or inhibit activation of the renin-angiotensin-aldosterone system. Preferred compositions contain paricalcitol with at least one of these other agents. Such combinations can avoid ACE inhibition escape and aldosterone escape with subsequent increase in angiotensin II and aldosterone generation.

Suitable ACE inhibitors, ARB and aldosterone blockers are commercially available. Suitable ACE inhibitors include, but are not limited to: captopril (commercially available under the tradename CAPOTEN from Mylan), enalapril (commercially available under the tradename VASOTEC from Merck), fosinapril (commercially available under the tradename MONOPRIL from Bristol Myers Squibb), benzapril (commercially available under the tradename LOTENSIN from Novartis Pharmaceuticals), moexipril (commercially available under the tradename UNIVASC from Schwarz Pharma), perindopril (commercially available under the tradename ACEON from Solvay), quinapril (commercially available under the tradename ACCUPRIL from Parke-Davis), ramipril (commercially available under the tradename ALTACE from Monarch), trandolapril (commercially available under the tradename MAVIK from Abbott Laboratories of North Chicago, Ill.), lisinopril (commercially available under the tradenames PRINIVIL from and ZESTRIL from Astra Zeneca).

Suitable angiotensin receptor blocking agents include, but are not limited to: losartan (commercially available as COZAAR from Merck), irbesartan (commercially available as AVAPRO from Bristol Myers Squibb and Sanofi), candesartan (commercially available as ATACAND from Astra Zeneca), eprosartan (commercially available as TEVETEN from Biovail Corporation of Canada), telmisartan (commercially available as MICARDIS from Boehringer Ingelheim) and valsartan (commercially available as DIOVAN from Novartis).

Suitable aldosterone blockers include, but are not limited to: eplerenone (commercially available under the tradename INSPRA from Pharmacia), spironolactone (commercially available under the tradenames Aldactone, Adultmin, Aldopur, Aldospirone, Almatol, Berlactone, Diatensec, Diram, Esekon, Hypazon, Idrolattone, Merabis, Novospiroton, Osiren, Osyrol, Pirolacton, Resacton, Sincomen, Spiractin, Spiroctan, Spirolacton, Spirolang, Spironex, Spirotone, Tevaspirone, Verospiron, Xenalon Lactabs, Youlactone).

Additional components, e.g., physiologically acceptable carriers, solvents, binders, antioxidants, colorants, substrates can be used as necessary or desired.

Preferred treatment or preventative regimens according to the present invention would administer therapeutically effective Vitamin D or Vitamin D analog-containing compositions according to the invention for a sufficient period to effect sustained or continuous delivery. As used herein, a “therapeutically effective dose” is a dose which in susceptible subjects is sufficient to prevent progression or cause regression of disease or which is capable of relieving the symptoms caused by the disease. For some disease conditions, pulsatile delivery may be less efficacious than continuous delivery.

An exemplary dosing regimen would provide the equivalent of about 0.5 micrograms of calcitriol per day or at least about 1 microgram calcitriol three times weekly. For paricalcitol, a suitable dosing regimen would provide the equivalent of about 4 micrograms paricalcitol daily or at least about 2 micrograms paricalcitol three times weekly administered as a bolus. Suitable dosing regimens for other Vitamin D or Vitamin D analogs, e.g., doxercalciferol, can be determined straightforwardly by those skilled in the art based on the therapeutic efficacy of the Vitamin D or Vitamin D analog to be administered.

Since ACEI, ARB and aldosterone inhibitors have different efficacies and affect the body through different pathways than Vitamin D does, compositions according to the present invention can incorporate an ACEI, ARB or aldosterone inhibitor to be administered according to conventional dosing regimens, which are well known and readily available to those skilled in the art.

The invention also contemplates continuous or sustained drug delivery forms containing the selected Vitamin D compound, and an ACEI and/or an ARB and/or an aldosterone blocker. Suitable delivery forms include, but are not limited to, tablets or capsules for oral administration, injections, transdermal patches for topical administration (e.g., drug to be delivered is mixed with a polymer matrix adhered to or absorbed on a support or backing substrate, e.g., ethylcellulose), depots (e.g., injectable microspheres containing the desired bioactive compounds) and implants. Techniques for making these drug delivery forms are well-known to those skilled in the art.

The present invention may be illustrated by the use of the following non-limiting examples:

### EXAMPLE I

### Activity of Paricalcitol to Suppress Renin Expression

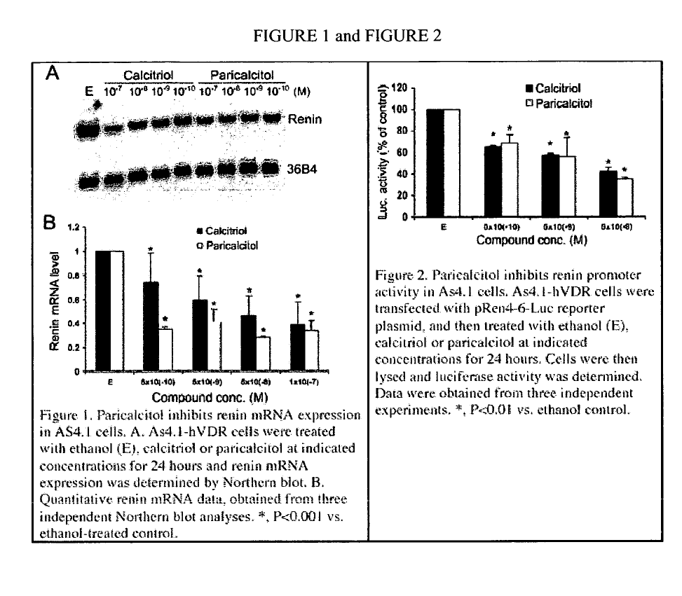

Using an in vitro system, an examination was carried out in order to determine the ability of paricalcitol to suppress renin expression. As shown in FIG. 1, based upon Northern blot analysis, paricalcitol treatment of As4.1-hVDR cells dose-dependently inhibits renin mRNA expression. In fact, its renin-inhibiting activity appears a bit more potent than calcitriol (FIG. 1A and B). This inhibitory activity is confirmed by renin promoter-luciferase assays, which examine the activity of paricalcitol to suppress renin gene transcription. In these assays, paricalcitol appears as least as potent as calcitriol in suppressing the activity of the renin gene promotor (FIG. 2).

### EXAMPLE II

### VDR Expression Profiles in Human, Normal Tissues

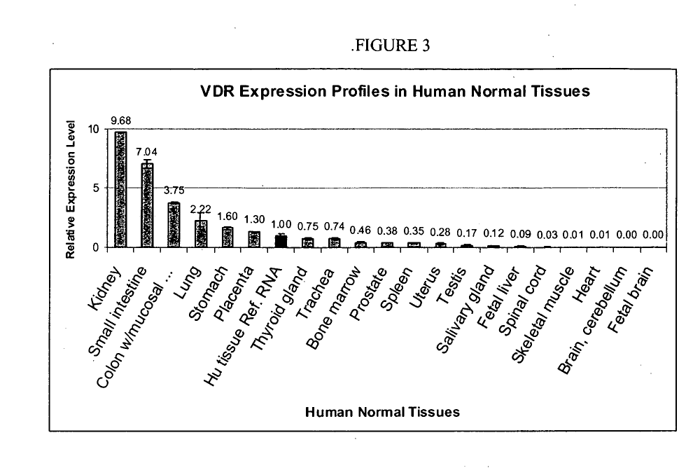

Due to the fact that (1) the leading cause of death for CKD patients is cardiovascular complications, (2) VDR activators (VDRAs) provide survival benefit for CKD patients, and (3) the effect is more profound for paricalcitol over calcitriol, it was hypothesized that VDRAs may affect the cardiovascular system. Since adult human cardiomyocytes do not express VDR (Q-PCR data shown in FIG. 3), likely the cardiovascular protective effects of VDRAs are on the functionality of smooth muscle cells and/or endothelial cells.

Relative expression was determined by quantitative PCR for Vitamin D receptor in various normal human tissues. The VDR expression level in each tissue was normalized to the VDR level in the reference pooled RNA sample.

### EXAMPLE III

### Effect of VDR Activators on PAI-1 Expression in Human Coronary Artery Smooth Muscle Cells

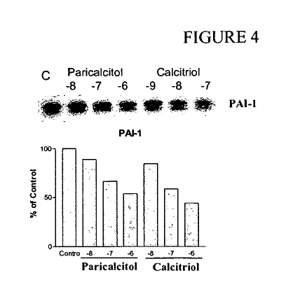

The effect of paricalcitol and calcitriol on PAI-1 in primary culture of human coronary artery smooth muscle cells was investigated. (See FIG. 4.) PAI-1 (plasminogen activator inhibitor type-1) is one of the risk markers for coronary heart disease, and is enhanced in atherosclerotic plague and co-localized with macrophages.

Human coronary artery smooth muscle cells were incubated with paricalcitol or calcitriol at the indicated concentration for 24 hr at 37° C. Samples were solubilized in SDS-PAGE sample buffer, and the protein content in each sample was determined by the Bio-Rad dye-binding protein assay. Samples were resolved by SDS-PAGE using a 4-12% gel, and proteins were electrophoretically transferred to PVDF membrane for Western blotting. The membrane was blotted for 1 h at 25° C. with 5% nonfat dry milk in PBS-T and then incubated with a mouse anti-PAI-1 monoclonal antibody in PBS-T overnight at 4° C. The membrane was washed with PBS-T and incubated with a horseradish peroxidase-labeled anti-rabbit antibody for 1 h at 25° C. The membrane was then incubated with detection reagent (SuperSignal WestPico). The specific bands were visualized by exposing the paper to Kodak BioMax films.

FIG. 4 shows the results from Western blot using an anti-PAI-1 antibody. Two observations may be noted in these studies: (1) 100% inhibition of growth was never achieved even at 1 μM of any of the test compound. Confocal microscopy studies confirm that, although these drugs are potent in inducing the translocation of VDR from cytoplasm to nucleus, not all cells respond to VDRAs even after 2 h of exposure, which may explain the <100% inhibition. (2) Although paricalcitol is known to be less potent than calcitriol in the clinical studies, it exhibits similar potency to calcitriol in this assay. By checking the effect of drugs on the expression of 24(OH)ase, it was found that paricalcitol is less potent than calcitriol on stimulating the expression of 24(OH)ase, which may partially explain the higher potency of paricalcitol in this assay.

These results show that paricalcitol and calcitriol are equally potent in reducing the PAI level in human coronary artery smooth muscle cells. Paricalcitol is usually dosed approximately 4 fold higher than calcitriol in the clinical situation, which may translate into a 4-fold higher potency in regulating the function of smooth muscle cells.

1. A sustained release pharmaceutical composition for preventing, treating and delaying progression of diseases caused by over-activation of the renin-angiotensin (II)-aldosterone system, comprising:
a therapeutically effective amount of Vitamin D or Vitamin D analog; and optionally a therapeutically effective amount of at least one member of the group consisting of an angiotensin converting enzyme inhibitor, an angiotensin (II) receptor 1 blocker, and an aldosterone blocker.

2. A sustained release pharmaceutical composition according to claim 1, wherein said Vitamin D or Vitamin D analog is selected from the group consisting paricalcitol, calcitriol and doxercalciferol.

3. A sustained release pharmaceutical composition according to claim 1 wherein said composition is in a transdermal patch form.

4. A sustained release pharmaceutical composition according to claim 1 wherein said composition is in an oral dosage form.

5. A sustained release pharmaceutical composition according to claim 1 wherein said composition is in a subcutaneous dosage form.

6. A sustained release pharmaceutical composition according to claim 1 wherein said composition is in an injectable dosage form.

7. A sustained release pharmaceutical composition according to claim 6, wherein said injectable dosage form is a member of the group consisting of a subcutaneous dosage form and a depot dosage form.

8. A sustained release pharmaceutical composition according to claim 5 wherein said composition is in an implantable form.

9. A pharmaceutical composition for treating, preventing or delaying progression of disease caused by over-activation of the renin-angiotensin (II)-aldosterone system in a mammal, comprising:
a therapeutically effective amount of Vitamin D or Vitamin D analog; and an optional therapeutically effective amount of at least one member of the group consisting of an angiotensin-converting-enzyme inhibitor, an angiotensin (II) receptor 1 blocker, and an aldosterone blocker.

10. A pharmaceutical composition according to claim 9, wherein said Vitamin D or Vitamin D analog is selected from the group consisting of paricalcitol, calcitriol, and doxercalciferol.

11. A pharmaceutical composition according to claim 9 wherein said composition is in the form of a transdermal patch.

12. A pharmaceutical composition according to claim 9 wherein said composition is in oral dosage form.

13. A pharmaceutical composition according to claim 9 wherein said composition is in a subcutaneous dosage form.

14. A pharmaceutical composition according to claim 9 wherein said composition is in an injectable dosage form.

15. A pharmaceutical composition according to claim 14 wherein said injectable dosage form is a member of the group consisting of a subcutaneous dosage form and a depot dosage form.

16. A pharmaceutical composition according to claim 15 wherein said composition is in an implantable form.

17. A method of preventing, treating and delaying disease progression of disease caused by over activation of the renin-angiotensin (II)-aldosterone system in a mammal, comprising the step of administering to said mammal a pharmaceutical composition according to claim 9.

18. A method according to claim 17, wherein the administering step is continuous.

19. A method according to claim 17, wherein the administering step is carried out using a transdermal patch.

20. A method according to claim 17, wherein the administering step is carried out using an oral dosage form.

21. A method according to claim 17, wherein the administering step is carried out using an injectable dosage form.

21. A method according to claim 17, wherein the administering step is carried out using a subcutaneous dosage form.
